(19) United States
(12) Patent Application Publication
(10) Pub. No.: US 2019/0096278 A1
(43) Pub. Date: Mar. 28, 2019

(54) ARCHITECTURE, SYSTEM AND METHOD FOR DYNAMIC USER SEMINARS, PROGNOSIS, AND MEASUREMENT

(71) Applicants: Jason YIM, San Diego, CA (US); Merle RICHMAN, La Jolla, CA (US)

(72) Inventors: Jason Yim, San Diego, CA (US); Merle W Richman, III, La Jolla, CA (US)

(73) Assignee: Chord Health, Inc., San Diego, CA (US)

(21) Appl. No.: 15/322,448

(22) PCT Filed: Aug. 15, 2014
(86) PCT No.: PCT/US2014/051381

§ 371 Date: Dec. 3, 2018

Related U.S. Application Data
(60) Provisional application No. 62/018,512, filed on Jun. 27, 2014.

(51) Int. Cl. G09B 19/00; G16H 70/20; G16H 40/20

(52) U.S. Cl. CPC G09B 19/00; G16H 40/20; G16H 70/20

(57) ABSTRACT

Embodiments of systems and methods that dynamically provide users with training/seminar presentations or direct users to attend specific training or seminars based on user demographics, other user demographics and results metrics. Embodiments of systems and methods that predict a user's potential progress when compliant or based on measured compliance with seminar participation based on other user demographics, other users' results metrics, health or training profession's analysis, and the user's result metrics before, after, and while participating in the seminars. Embodiments of systems and methods that determine a user's results or metric based on a combination of inputs including user subjective and objective data or input and health or training profession's analysis. Other embodiments may be described and claimed.

### TECHNICAL FIELD

Various embodiments described herein relate generally to dynamic user training/seminar presentation, user training/results prognosis, and user result metric apparatus, systems, and methods.

### BACKGROUND INFORMATION

It may be desirable to dynamically provide users with training/seminar presentations or direct users to attend specific training or seminars based on user demographics, other user demographics, a health or training professional's (HTP) analysis, and results metrics. It may be further desirable to predict a user's potential progress when compliant or based on measured compliance with seminar participation based on other user demographics, other users' results metrics, HTP analysis, and the user's result metrics while participating in the seminars. It may be further desirable to determine a user's results or metric based on a combination of inputs including user subjective and objective data or input. The present invention provides such activities and data.

### BRIEF DESCRIPTION OF THE DRAWINGS

FIG. 1 is a block diagram of web based dynamic user training, prediction, and result (DUSTPR) architecture according to various embodiments.

FIG. 2A is a block diagram of a local web based DUSTPR architecture according to various embodiments.

FIG. 2B is a block diagram of DUSTPR administration and multiple electronic training dynamic training system architecture according to various embodiments.

FIG. 3A is a diagram of user login/setup, seminar presentation, and feedback communication between a user device and a dynamic training system (DTS) in DUSTPR architecture according to various embodiments.

FIG. 3B is a diagram of communication between a user activity monitor module (UAMM) and a dynamic training system (DTS) in DUSTPR architecture according to various embodiments.

FIG. 3C is a diagram of a health or training professional (HTP) login/setup, user or group selection, and reporting for user/group communications between a HTP device and a dynamic training system (DTS) in DUSTPR architecture according to various embodiments.

FIG. 4A is a block diagram of DUSTPR architecture providing a user setup/login/prediction web page according to various embodiments.

FIG. 4B is a block diagram of DUSTPR architecture providing a seminar/training calendar-selection web page according to various embodiments.

FIG. 4B is a block diagram of DUSTPR architecture providing seminar media via a web page according to various embodiments.

FIG. 4C is a block diagram of dynamic training system architecture providing a media display web page according to various embodiments.

FIG. 4D is a block diagram of DUSTPR architecture providing seminar feedback web page according to various embodiments.

FIG. 4E is a block diagram of DUSTPR architecture providing a health or training professional (HTP) user or group report/study web page according to various embodiments.

FIG. 5 is a block diagram of a dynamic training system according to various embodiments.

FIG. 6A is a flow diagram illustrating several methods according to various embodiments.

FIG. 6B is a flow diagram illustrating several methods according to various embodiments.

FIG. 7A is a block diagram of an article according to various embodiments.

FIG. 7B is a block diagram of an article according to various embodiments.

FIG. 8A is a simplified diagram of a various training or seminar modules of a DTS according to various embodiments.

FIG. 8B is a simplified diagram of a first user specific seminar or training calendar including one or more designated training or seminar modules according to various embodiments.

FIG. 8C is a simplified diagram of a second user specific seminar or training calendar including one or more designated training or seminar modules according to various embodiments.

### DETAILED DESCRIPTION

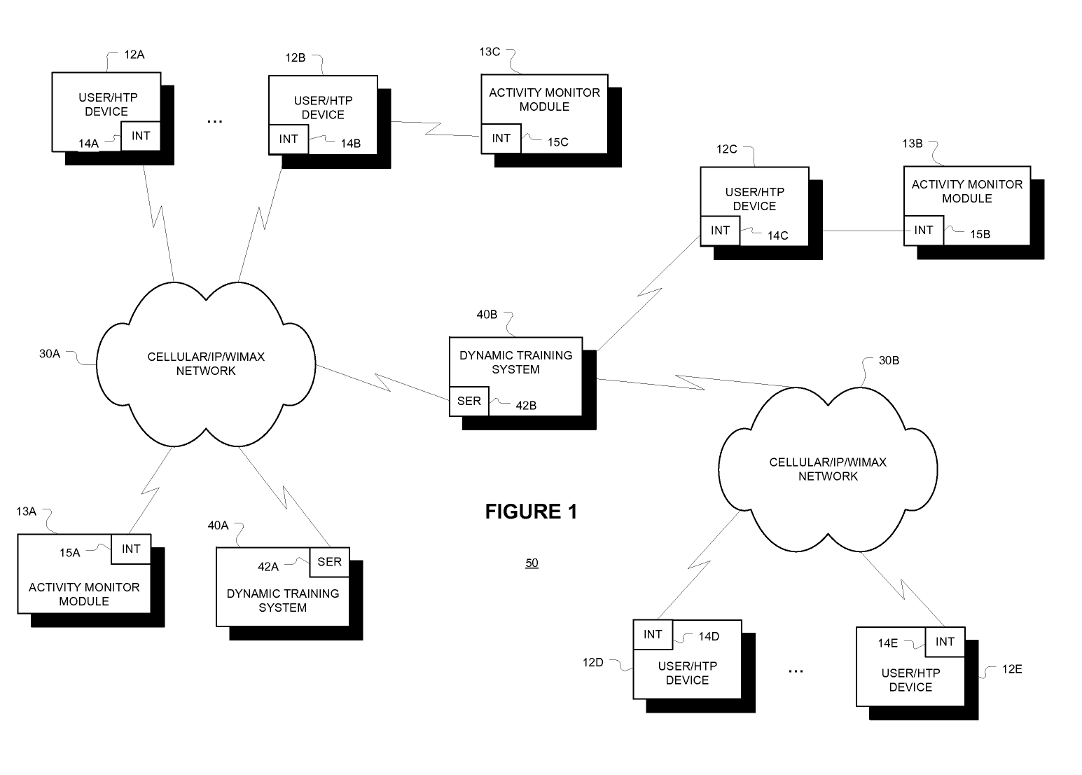

FIG. 1 is a block diagram of a dynamic user training, prediction/prognosis, and results/metric (DUSTPR) architecture 50 according to various embodiments. As shown in FIG. 1, DUSTPR architecture 50 includes a first and a second dynamic training system (DTS) 40A, 40B, several networked user/heath or training professional (HTP) devices (UHD) 12A-E, and wired/wireless networks 30A, 30B. In an embodiment, a dynamic training system (DTS) 40A may be a web based system that may communicate with user/HTP devices (UHD) 12A-E via a wired or wireless connection. 30A, 30B

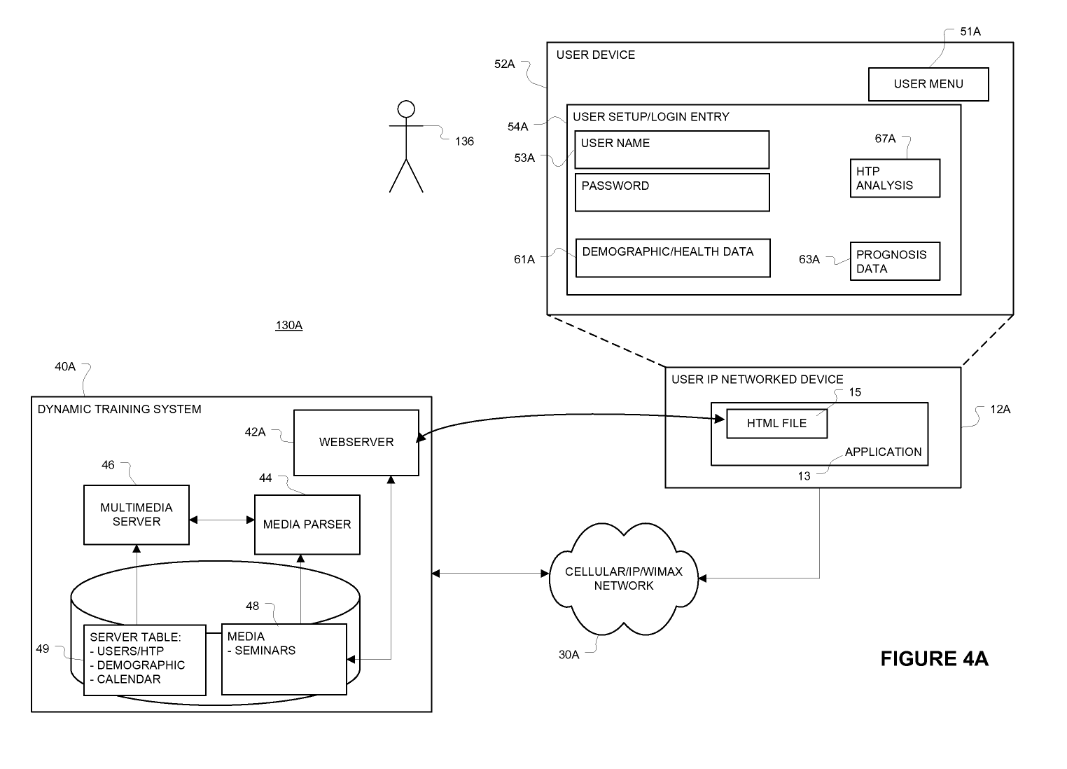

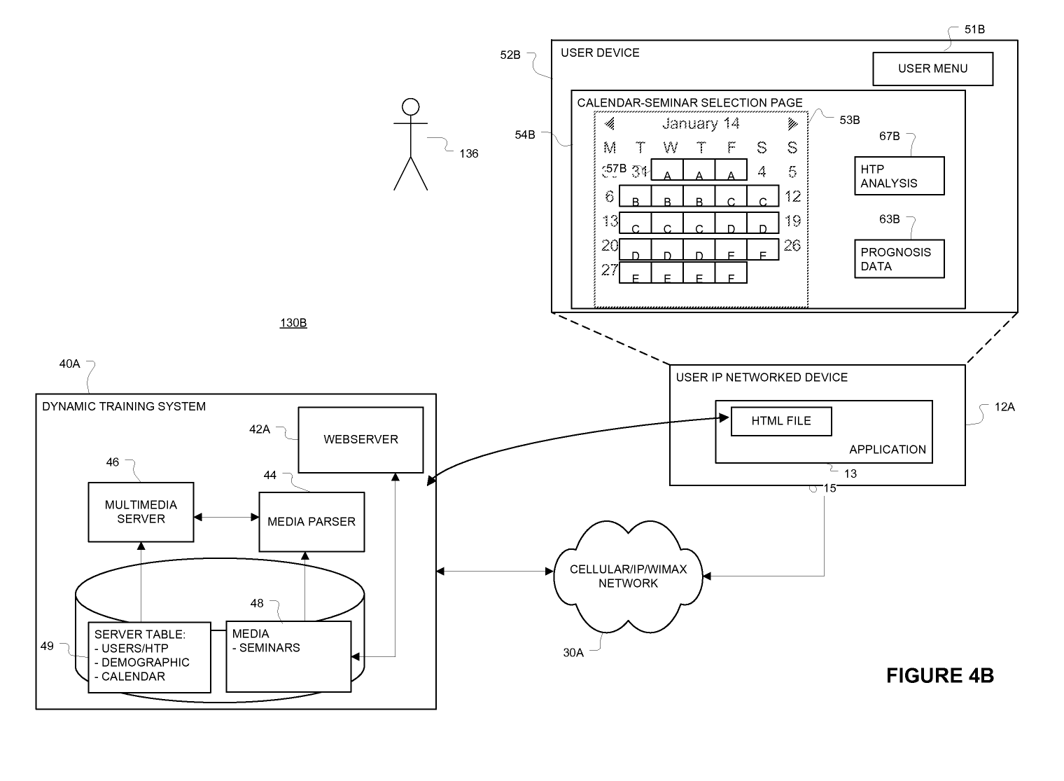

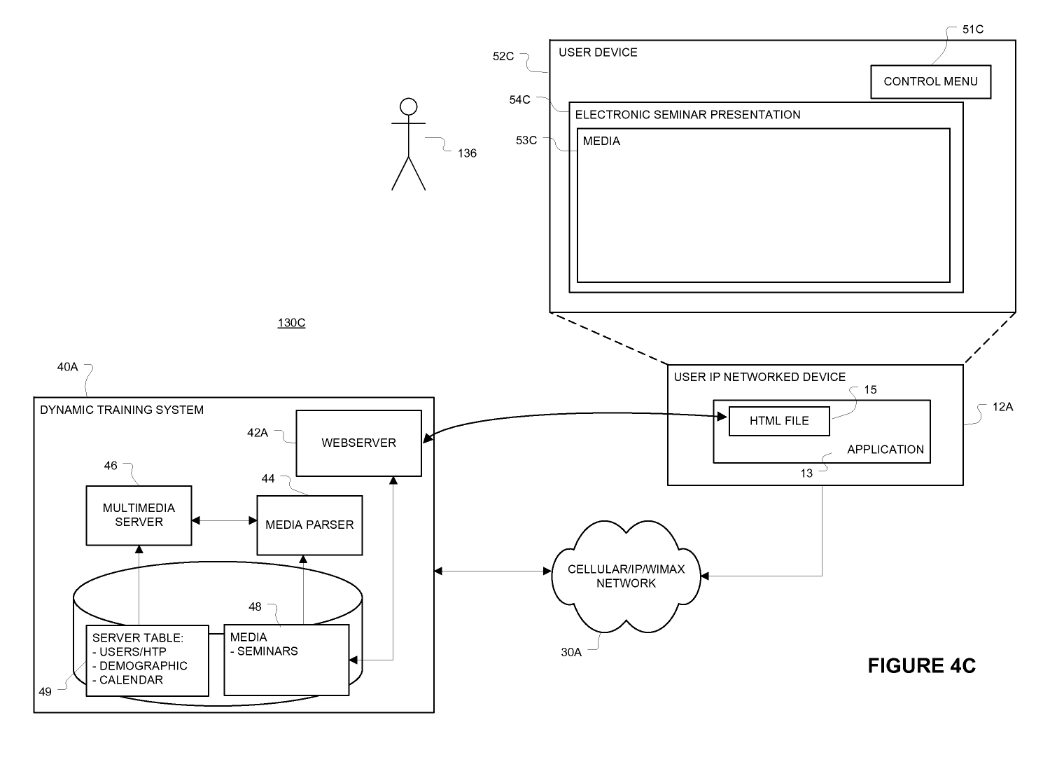

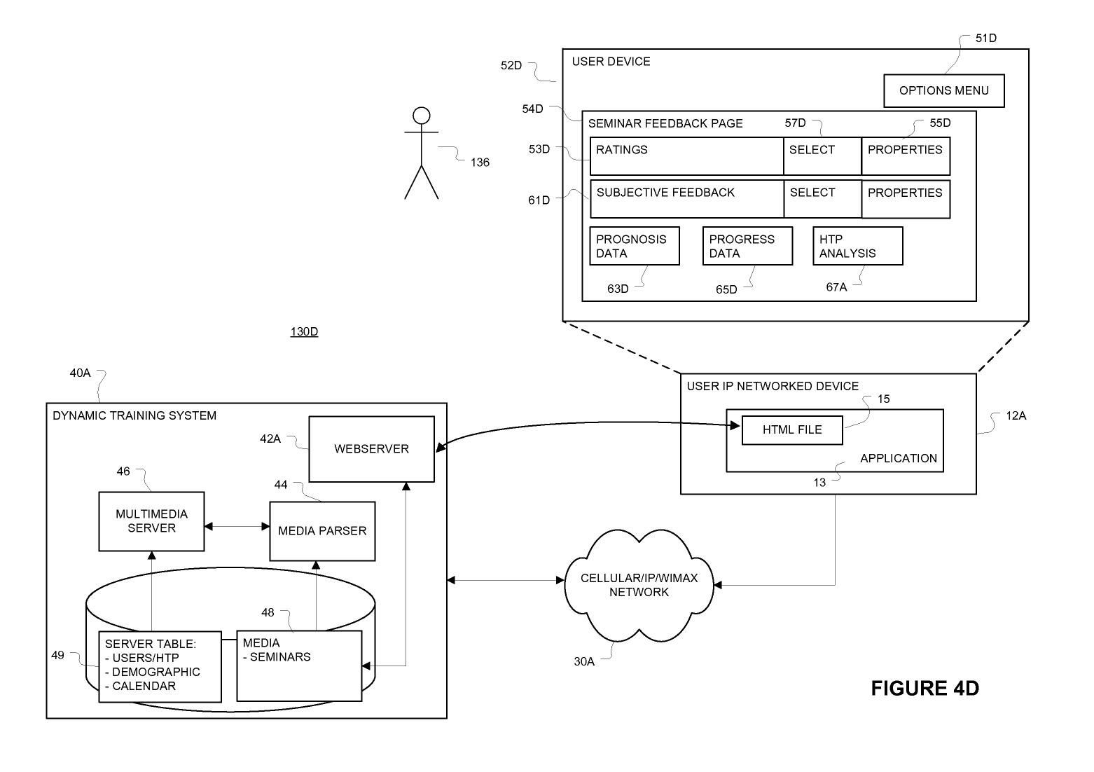

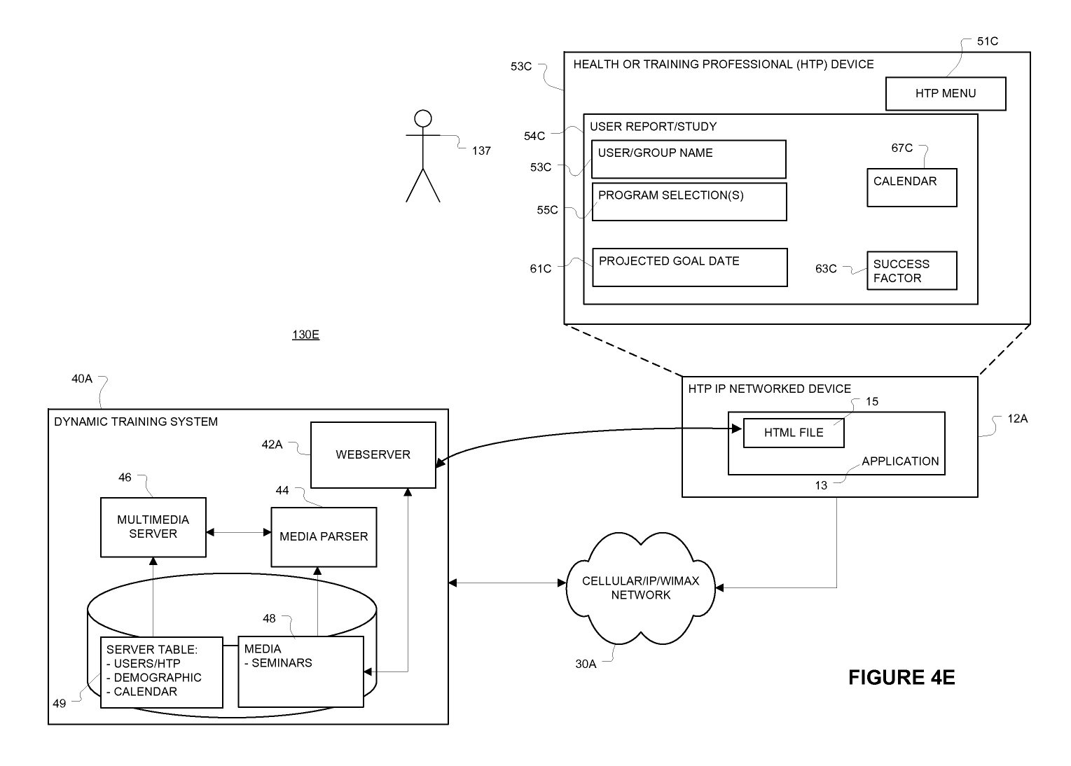

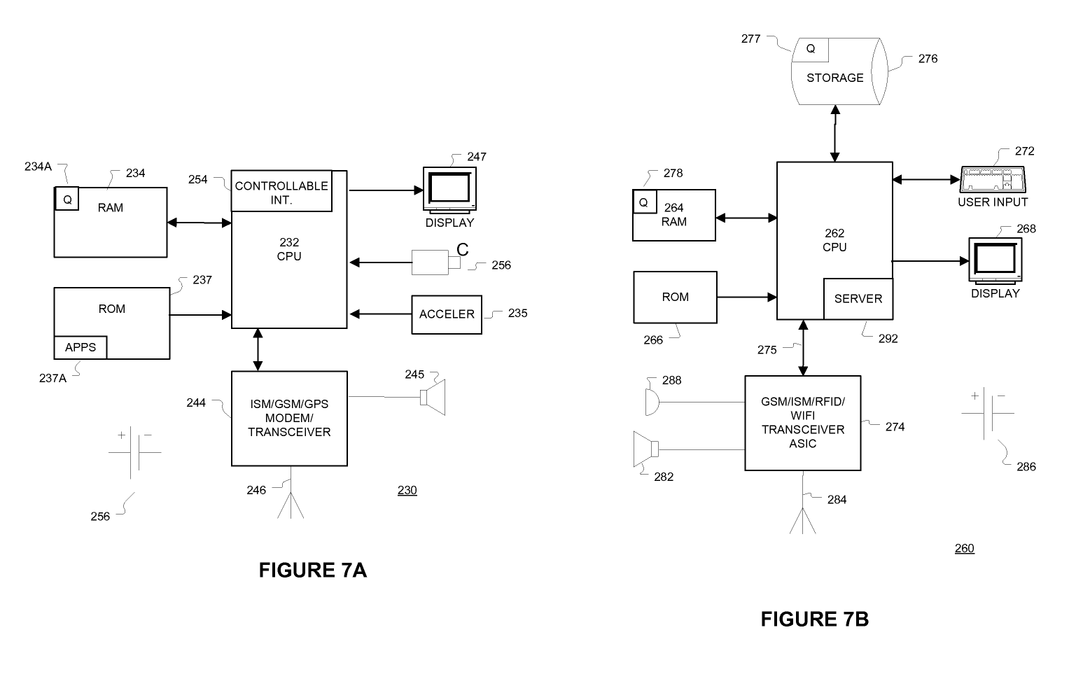

A DTS 40A, 40B may include a web server 42A, 42B that may enable communication between a DTS 40A, 40B, a plurality of IP networked user/HTP (UHD) 12A-E, and user activity monitor modules (UAMM) 13A-13C. A DTS 40A, 40B may employ an application specific integrated circuit (ASIC) (274 FIG. 7B) to transceive signals with one or more UHD 12A-E or UAMM 13A-13C. A DTS 40A, 40B server 42A, 42B may be a webserver that communicates data that may be processed by a web browser application resident on a UHD 12A-E. In an embodiment, a DTS 40A, 40B server 42A, 42B may generate Hyper Text Markup Language (HTML) encoded data that a UHD 12A-12E may process via a resident web browser (13 FIGS. 4A-4E).

In an embodiment, a DTS 40A, 40B server 42A, 42B may communicate data including user data, group data, media, login, calendar, prediction/prognosis, feedback, and other data using an IP protocol or another protocol including an application specific protocols. A UHD 12A-12E may include a program to decode/encode the application specific protocol communications between the UHD 12A-12E and a DTS 40A, 40B. As shown in FIG. 1, a UHD 12A, 12B may be coupled a DTS 40A, 40B via a network 30A. The network 30A may be a local network or a network of networks. A UHD 12C may be coupled directly to a DTS 40B via a wired or wireless connection. In another embodiment, a UHD 12D, 12E may communicate with a DTS 40B via a network 30B.

A UHD 12A-12E may also communicate with UAMM 13A-13C and forward activity data to a DTS 40A, 40B, another UAMM 13A-13C, or UHD 12A-12E. In an embodiment, a UAMM 13A-13C may communicate activity data for a user directly to a DTS 40A, 40B, another UAMM 13A-13C, or UHD 12A-12E using a wired, wireless, or direction connection 30A, 30B. In a further embodiment, a UHD 12A-12E may also provide user activity data to a DTS 40A, 40B, an UAMM 13A-13C, or another UHD 12A-12E via a wired, wireless, or direction connection 30A, 30B. The user activity data may include accessing seminar/training media, attending seminar/training events, and performing activities represented or shown in a seminar/training media. A UHD 12A-12E and UAMM 13A-13C may include one or more sensors that may determine physical activity or health status (blood pressure, heart rate, glucose level, and others) of a user 136. The sensors may include biometric sensors, accelerometer, global positioning system (GPS) module, and other location or sensor modules. The biometric sensors may be able to detect a user's mental and physical activity levels and health status levels in an embodiment.

A DTS 40A may communicate media and data with another DTS 40B. A DTS 40A may be a central or main DTS 40A, 40B that controls the operation of other DTS 40A, 40B in architecture 50. A UHD 12A-12E and UAMM 13A-13C may include an interface (network interface controller (NIC)) 14A-14E, 15A-C that enables IP based communication with a DTS 40A, 40B, another UHD 12A-12E, or UAMM 13A-13C. The interfaces 14A-14E, 15A-15C may include a modem/transceiver 244 (244, FIG. 7A). The modem/transceiver 244 may include an application specific integrated circuit (ASIC). A network 30A, 30B may be a local network, a network of networks, or a worldwide network of networks, termed the “Internet”, cellular network, or WiMax networks. In an embodiment, a UHD 12A-E or UAMM 13A-C may communicate with a DTS 40A, 40B via several networks. It is noted that in an embodiment, the networks 30A, 30B may be industrial, scientific and medical (ISM) radio bands, Groupe Special Mobile (GSM), Code-division multiple access (CDMA), time division multiple access (TDMA), mesh, and short messaging system (SMS) based network, WiMax, IP (wired or wireless network) such as 802.11a, b, g, n networks.

A network 30A, 30B may be a terrestrially based network or satellite based network, or combination thereof. Each UHD 12A to 12E may include an interface 14A to 14E that enables communication between a UHD 12A-12E and a DTS 40A, 40B, other UHDS 12A-12E, and UAMM 13A-C via a network 30A, 30B directly or indirectly. Each UAMM 13A to 13C may include an interface 15A to 15C that enables communication between a UAMM 13A-C and a DTS 40A, 40B or UHD 12A-12E, or other UAMM 13A-C via a network 30A, 30B directly or indirectly. As noted a UAMM 13A-C may also communicate directly with a UHD 12A-12E, other UAMM 13A-C, or DTS 40A-40B using a wired or wireless communication protocol. In an embodiment, a UHD 12A-E may be cellular device such an iPhone® or other smartphone, tablet device including an iPad®, laptop, tablet, desktop, television, gaming console (Xbox, Xbox 360, Xbox One, PS2, PS3, PS4, Nintendo Wii), steaming module (Roku, Chromecast, Apple TV) or other electronic device capable of communicating via one or more wired or wireless protocols and capable of viewing web pages or other media presentation formats. In an embodiment, a DTS 40A, 40B may be an electronic device 260 that may include a module 274 to communicate signals with a UHD 12A-12E, other DTS 40A, 40B, or UAMM 13A-13B. A DTS 40A, 40B may also include a web or presentation server 42A, 42B (292, FIG. 7B).

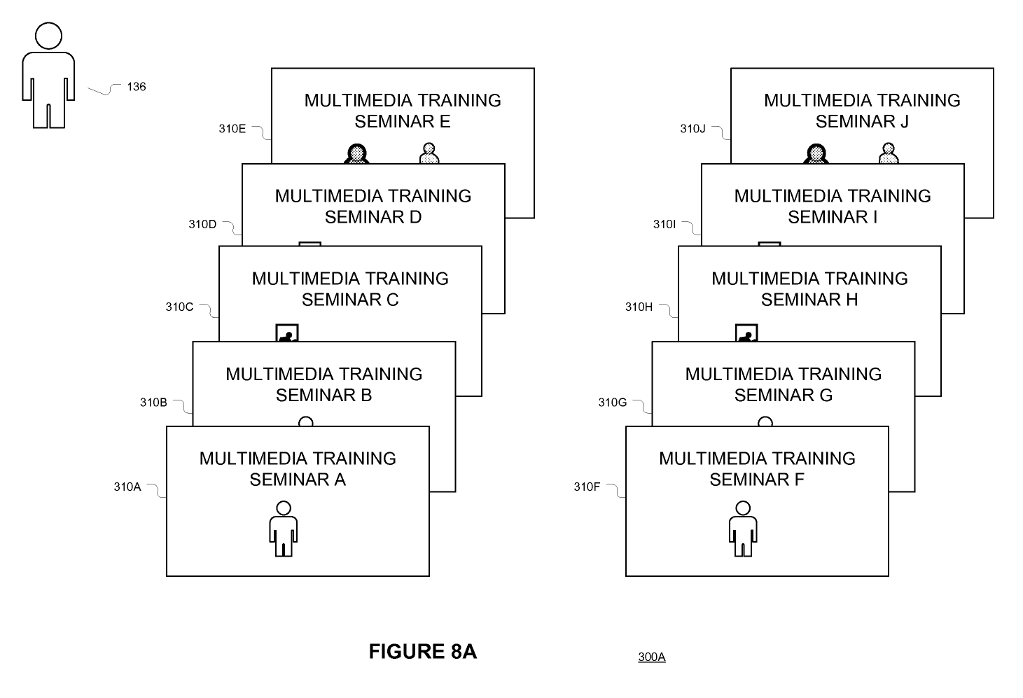

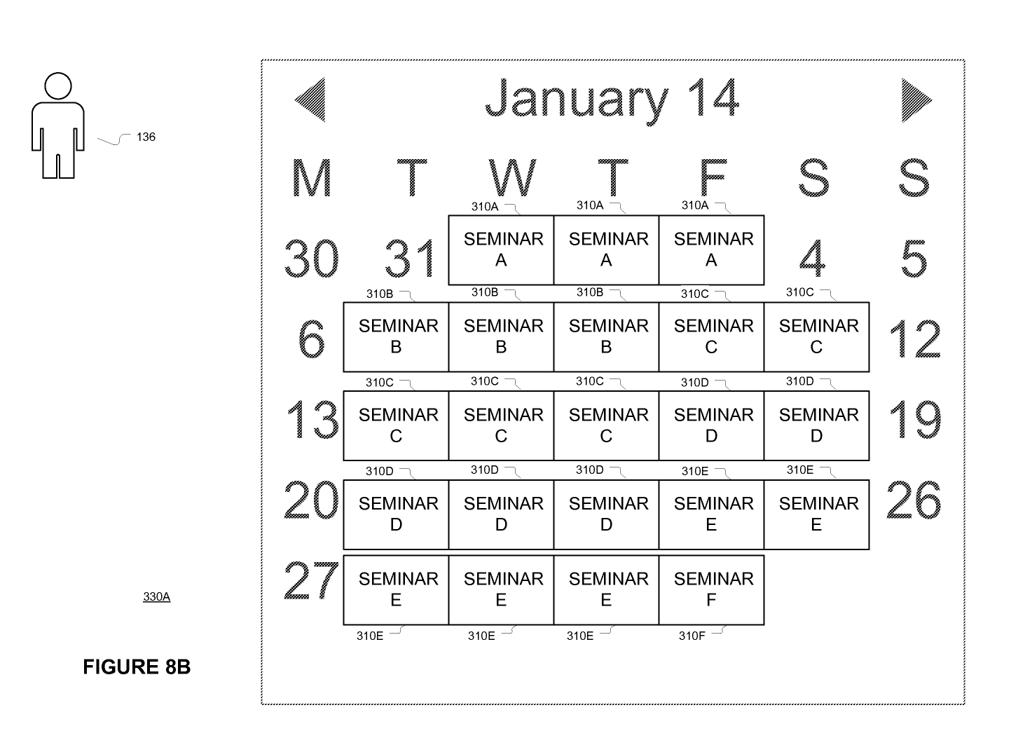

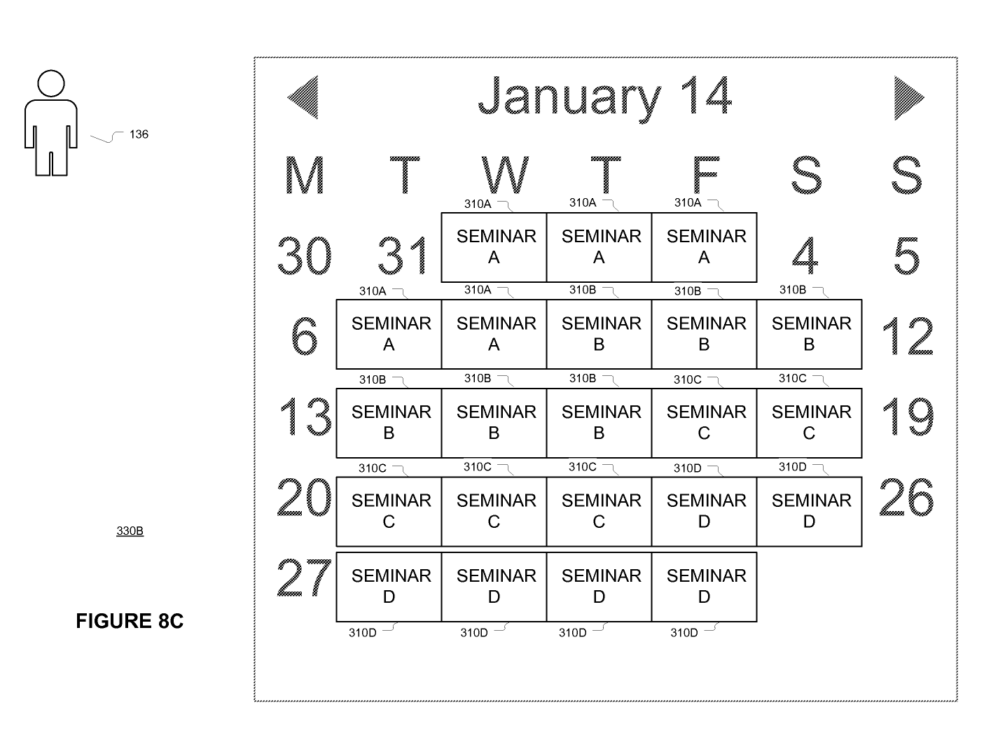

In an embodiment, a user/HTP 12A-12E may receive digital media content (310A-J, FIG. 8D) from a DTS 40A-40B server 42A-42BA in response to a request from the UHD 12A-12E (to a DTS 40A-40D). The digital media content 310A-J may include video, audio, pictures, text, or any combination thereof. In an embodiment, architecture 50 may enable a user 136 or HTP 137 to receive seminars or training events selected based on various user parameters, learn about the seminars and events, and find a time/date of a seminar or training event. Architecture 50 may also predict the effects of a user or user group participating in or more seminars or training events 310A-310J. Architecture 50 may also provide a feedback system that enables a user to provide a subjective effect of the user attending or participating in a seminar or training event 310A-310J. In an embodiment, architecture 50 may monitor a user's 130 activity during, before, or after attending or participating in one or more seminars 310A-310J.

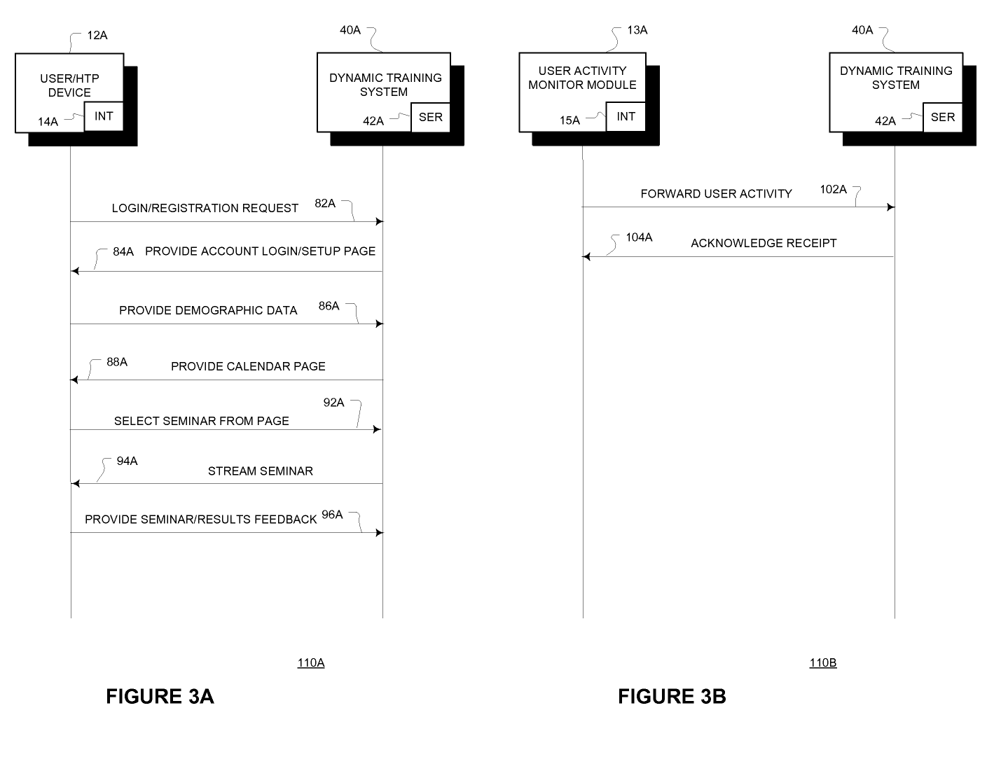

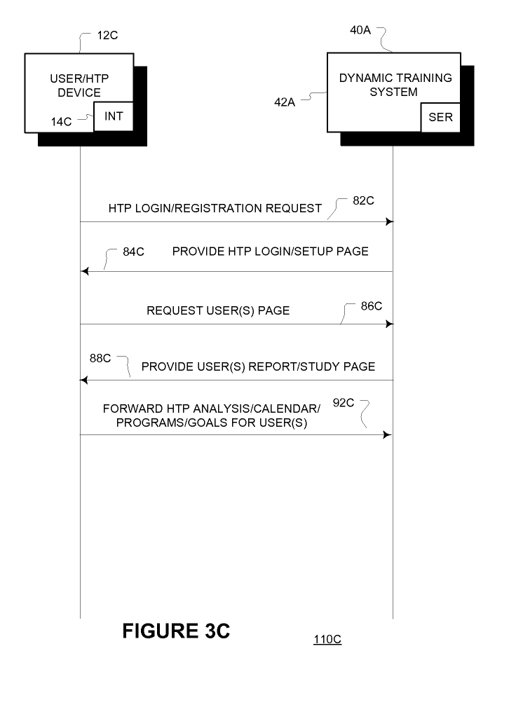

Architecture 50 via a DTS 40A, 40B may also enable a health or training professional (HTP) 137 to recommend or prescribe one or more seminars or events. A HTP 137 via a UHD 12A-12E may login to a DTS 40A, 40B (communications, 82C, 84C of FIG. 3E) and request a user or user group page study or report page (web page 53C shown in FIG. 4E, communication 86C of FIG. 3C). A HTP 137 may use a dedicated email address for the user, user group, or the HTP to assign one or more programs for the user or user group. A HTP 137 may also use a short messaging service (SMS) number or address for the user, user group, or the HTP to assign one or more programs for the user or user group.

Architecture 50 via a DTS 40A, 40B may also enable a health or training professional (HTP) 137 to provide reports, studies, progress, and other data for a user or user group. A HTP 137 via a UHD 12A-12E forward reports, studies, progress, and other data for a user or user group via a DTS 40A, 40B (web page 53C shown in FIG. 4E, communications 88C, 92C of FIG. 3C). A HTP 137 may use a dedicated email address for the user, user group, or the HTP to provide reports, studies, progress, and other data for a user or user group. A HTP 137 may also use a short messaging service (SMS) number or address for the user, user group, or the HTP to provide reports, studies, progress, and other data for a user or user group. As shown in FIG. 4E, the user or user group study/report data may include program, seminar, or training selections/prescriptions/recommendations 55C, a user or user group projected goal achievement data 61C, calendar for program, seminar, or training selections/prescriptions/recommendations 67C, a user or user group success factor (progress toward their goal in an embodiment) 63C.

The user or user group data may be stored in the server table 49 of a DTS 40A, 40B as shown in FIGS. 4A-4E. A HTP's user or user group seminar, or training selections/prescriptions/recommendations 55C, user or user group projected goal achievement data 61C, calendar for program, seminar, or training selections/prescriptions/recommendations 67C, and a user or user group success factor (progress toward their goal in an embodiment) 63C may also be stored in the server table 49 of a DTS 40A, 40B as shown in FIGS. 4A-4E. As noted a HTP may provide such data via a web page or other electronic mechanism in an embodiment. In an embodiment, a UHD 12A-12E may have a HTP specific program stored or executed by the UHD 12A-12E to enable a HTP to provide such data.

In an embodiment, a HTP 137 via a UHD 12A-12E and DTS 40A, 40B may receive user or user group data. The user or user group data may include user(s) subjective or objective activity data, including seminars or training programs attended, viewed, or attempted. The data may also include UAMM 13A-13C collected before, during, or after user(s) attend, view, or attempt one or more seminars or training programs. The data may be presented in graphical formats and include video/audio of user(s) attending, viewing, or attempting one or more seminars or training programs.

In an embodiment, the seminars or training modules 310A-310J may be programs may be designed to affect one or more user's (user or user group) 136 physical or mental characteristics. In an embodiment, the training modules 310A-310J may be physical exercises to be performed by a user or user group. A DTS 40A-40B may stream or communicate digital media representing or directing the physical activities to be performed. A DTS 40A-40B may also provide times and locations of where the seminar or training is shown live or broadcast or displayed by an electronic media device. In an embodiment, a new user or group of a DTS 40A, 40B may register with or login to the DTS 40A-40B via a UHD 12A-12E. A user 136 may provide one or more demographic details and select a seminar series or indicate a physical attribute or issue they would like to address, e.g., a sore lower back. In an embodiment, a DTS 40A, 40B may ask for specific user demographic information as a function of the seminar or training series or program elected or the physical or mental issue the user would like to address or that has been assigned to the user by a 3^{rd} party, including a HTP.

The user may receive or employ a UAMM 13A-13C or load a program on their UHD 12A-12E to monitor certain activities related to the training modules 310A-310J. The monitored activities may provide objective user data that may correlate to the user's progress or improvement (of a physical or mental issue or characteristic). In an embodiment, a user 136 via a UHD 12A-12E may also provide subjective feedback via one or more questions provided by a DTS 40A, 40B. In an embodiment, a DTS 40A, 40B may predicts a user's potential physical or mental characteristic change based on their provided demographics and the selected, elected, or prescribed seminars or training modules 310A-310J.

The demographics may include age, height, weight, other body measurements, other health issues or conditions (diabetic or other condition), geographical location, marital status, occupation, and generic characteristics (including race) or other generic specific factors. A DTS 40A, 40B may develop or employ a database of related or similar users (based on demographics) to predict an outcome or user prognosis. A DTS 40A, 40B may also use other user activity data in part to predict an outcome or user prognosis. A DTS 40A, 40B may not provide other use specific identifiers to protect other user's privacy. A DTS 40A, 40B may employ avatars or other mechanisms to protect a user's identity while enabling a DTS 40A, 40B to correlate related users based on demographics and training modules 310A-310J.

In an embodiment, when a user 136 via a UHD 12A-12E forwards a request for a seminar or training module 310A-J, a DTS 40A-40B may respond with an account login/setup page or associated media content selection page (52C, FIG. 4C) or calendar page (52B, FIG. 4B) for live or future seminar or training module. A DTS 40A-40B may forward the associated media content selection page (52C, FIG. 4C) or calendar page (52B, FIG. 4B) when the UHD 12A-12E user is known or can be determined based on the received request and is registered with the DTS 40A-40D. In another embodiment, a DTS 40A-40B server 42A-42B may always forward an account setup (or login page for a registered user) for any request based on system security protocols.

In an embodiment, a DTS 40A, 40B user or administer may associate digital media content 48 with a seminar or training module 310A-J. In an embodiment, a DTS 40A, 40B may receive the associated digital media content and transcode via the media parser 44 (FIG. 4A) the content into different formats that may be required for the content to be viewable on various UHD 12A-12E platforms.

In an embodiment, associated media content may include video content. A DTS administrator may generate the video content and upload the video content to a DTS 40A, 40B via a web browser application. The digital video content my be device specific or another video encoding format, such as a version of H.264, MPEG, AVI, WMV, H.265, or other digital video format. Further, the video resolution and audio encoding may vary by user selection.

A DTS 40A-40B may convert a stored digital video content or media to one or more standard digital formats having one or more resolutions, and audio encoding based on a user's 130 requesting device 12A-E or profile. Accordingly, a user may be able to associate particular media format with their profile and view the content using standard software-algorithms on their respective media devices 12A-12E.

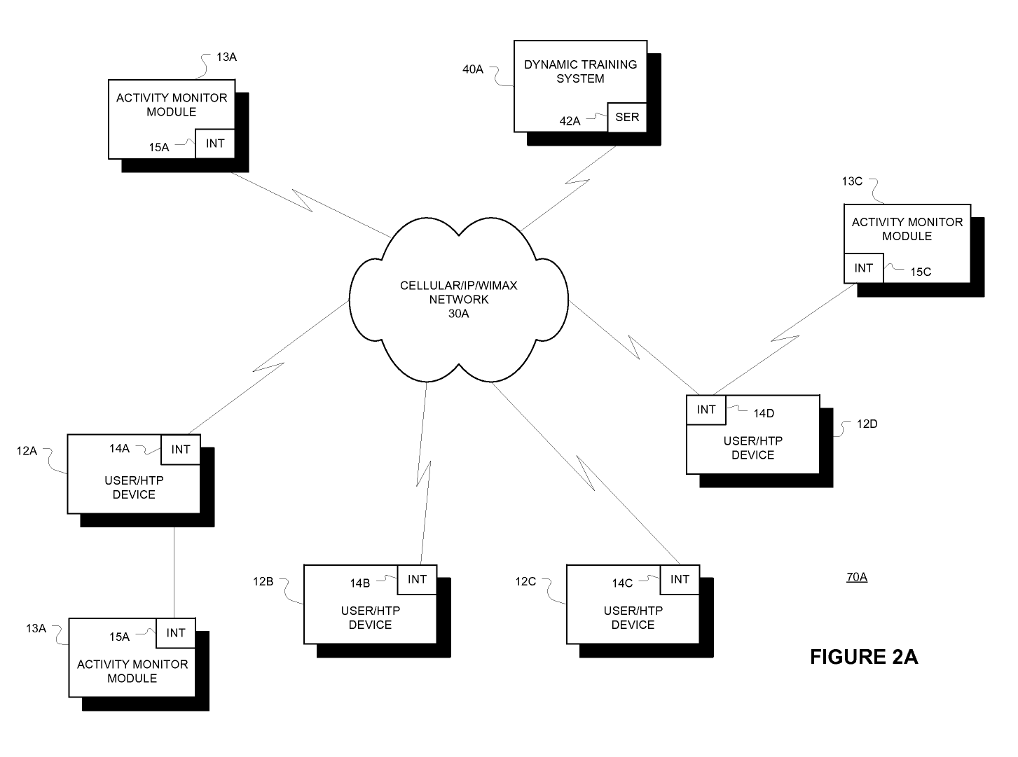

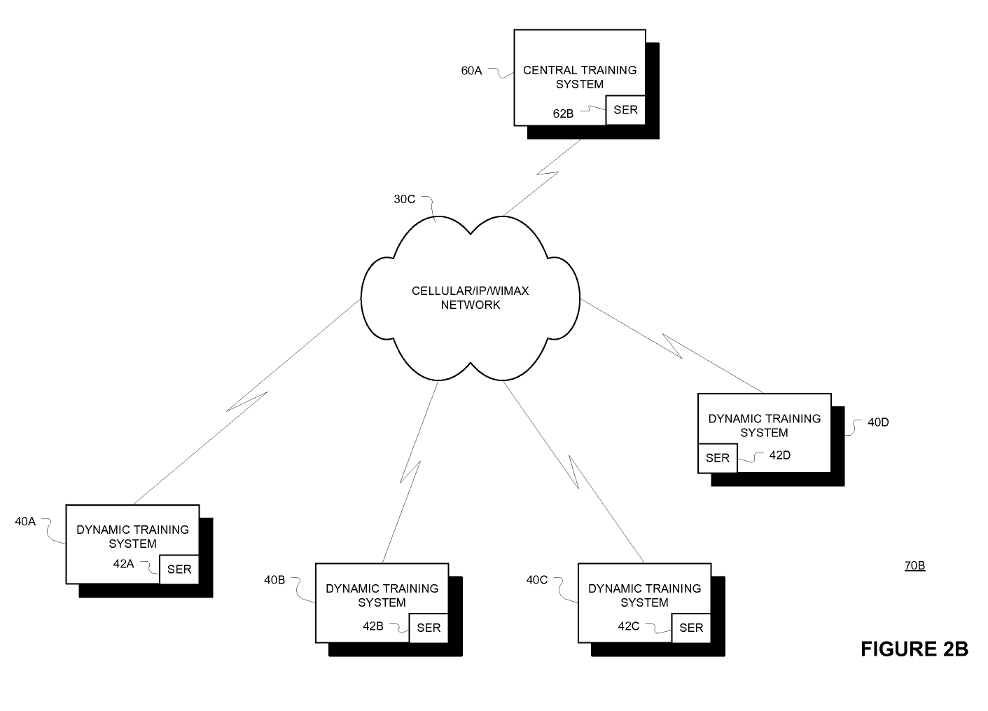

FIG. 2A is a block diagram of a local web based image associated dynamic content (DUSTPR) architecture 70A according to various embodiments. As shown in FIG. 2A, architecture 70A may include a DTS 40A couplable to a plurality of UDs 12A-12D via a network 30A. The network 30A may be a local network or a network of networks in an embodiment. The network 30A may include one more wireless communication devices include a wireless router, hub, and an Apple® airport express. FIG. 2B is a block diagram of administration and multiple dynamic training system architecture 70B according to various embodiments.

As shown in FIG. 2B, architecture 70B may include a DTS administration processing system (DAPS) 60A couplable to a plurality of DTSs 40A-40D via a network 30C. The network 30C may be a local network or a network of networks in an embodiment. In an embodiment, a DAPS 60A may correlate or duplicate server 42A-42D content (including databases 48, 49, FIGS. 4A-4E) across multiple DTS 40A-40C. Architecture 70B may employ multiple DTS 40A to 40D to reduce system lag for UDs 12A-12E located at different locations in a network or network of networks 30C. When a change is made at a first DTS 40A-40D, the DAPS 60A may propagate the change to other DTS 40A-40D including digital media content, user, and demographics, activity data, and feedback data (or other changes to databases 48, 49).

FIG. 3A is a diagram of communications between a user 136 via a UHD 12A and a DTS 40A in DUSTPR architecture according to various embodiments. Via a UHD 12A, a user 136 may generate a login request 82A and send the request to a DTS 40A via its NIC 14A and a network 30A, 30B. Via the network 30A, 30B, IP protocols, and its transceiver 244 (FIG. 7A), a DTS 40A may receive the request. In an embodiment, the request may be a web based request. A DTS 40A-B may employ the algorithm 170A shown in the FIG. 6A upon receipt of a login request (activity 172A). Upon receipt of the login request, a DTS 40A may search a user database or table in a server table (49, FIG. 4D) to determine if the user is an active or registered user.

When the user is not registered or active in the DTS 40A, the DTS 40A may generate and forward a user setup webpage (52A, FIG. 4A) to the requesting UHD 12A (activity 178A, FIG. 6A). As shown in FIG. 4A, the webpage 52A may include entries for a username 53A, password, and demographic/health data 61A. As noted, a user 136 may be registered with a DTS 40A or DTS 60A by a medical professional. The medical professional or the user may provide requested demographical/health data 61A. In an embodiment, a DTS 40A may provide different login pages 52A as a function of the requesting page. For example, a user's request (via a UHD 12A) may be website specific where various seminars or training modules are associated with different websites, organizations, individuals, or medical professionals.

In such an embodiment, the requested demographic/health data 61A may vary by the related or elected seminars or training modules. In another embodiment, a DTS 40A, 40B may assign one or more seminars or training modules 310A-J based on the user's registration (or registrar) and provided demographic/health data 61A. In an embodiment, a DTS 40A, 40B may store the demographic data and update the demographic database (activity 174B of process 170B of FIG. 6B). As shown in webpage 52A, a DTS 40A may calculate and provide a predicted result or completion date 63A for a user. A DTS 40A may predict a user's status change and timeline based on the user provided demographic/health data and selected or suggested seminar or training module 310A-J. A user's prognosis may be calculated based on the user's demographics, selected or suggested seminar/training modules 310A-J, other correlated users, the user's compliance or completed seminars to date, UAMM 13A-C data, and users' feedback. A user's prognosis may change as a user progresses through one or more seminars/training modules, UAMM user data is compiled, other correlated data is received, user feedback data is provided, and other user feedback data is compiled.

A DTS 40A may suggest a seminar/training module and schedule for a user 136 (activity 182A of FIG. 170A). FIG. 4B is a block diagram of DUSTPR architecture 130B providing a seminar/training calendar-selection web page 52B according to various embodiments. As noted a DTS 40A, 40B may generate and forward calendar-selection web page 52B based on the user's demographics, selected or suggested seminar/training modules 310A-J, other correlated users, the user's compliance or completed seminars to date, UAMM 13A-C data, and users' feedback. FIGS. 8B and 8C are simplified diagrams of user specific seminar or training calendars including one or more designated training or seminar modules according to various embodiments. As shown in FIG. 8B, a first user may assigned seminars A-F during a time period, while a second user may be assigned only seminars A-D during the same or similar time period. As noted, a user's suggested seminars or training modules 310A-J may be varied based on many factors including related users past or current performance as compiled in DTS 40A, 40B.

FIG. 6B is a flow diagram 170B illustrating several methods that may be employed by a DTS 40A, 40B to modulate user activities according to various embodiments. As shown in FIG. 6B, when new demographic or health data is provided or received for a user or correlated users, the user's or users' demographical database may be updated (activity 174B). A DTS 40A, 40B may use the demographical database 49A and other data to determine seminar/training selection and timing (calendar formation or modification). A DTS 40A-B may form or modify a user's calendar and prognosis based on a user's feedback data (activity 176B, 178B).

FIG. 4D is a block diagram of DUSTPR architecture 130D providing a seminar feedback web page 52D according to various embodiments. As shown in FIG. 4D, a DTS 40A may include a multimedia server 46, a media parser 44, a webserver 42A, a media database 48, and a server table/database 49. The server table/database 49 may store and compile users, users' demographics including activity and feedback, and user calendars. The webserver module 42A may generate web pages and receive web based requests. The multimedia server 46 may provide requested media including seminars from the media database 48. The media parser 44 may modify stored media including changing its format. A UHD 12A-E may include an application 13 that may process hypertext markup language (HTML) files to display the seminar feedback page 52D. The application 13 may be a web browser in an embodiment.

As shown in FIG. 4D, the feedback webpage 52D may include a ratings section 53D, a subjective feedback section 61D, a prognosis data window 63D, and a progress data window 65D. The rating and subjective sections 53D, 61D may include various selections 57D and properties 55D. A user 136 via the webpage 52D may be able to rate the quality or effectiveness of a particular seminar 310A-J. In an embodiment, a user may be presented with the seminar feedback webpage 52D after completing or reporting to have completed a seminar or training module 310A-J. A user 136 may also provide subjective feedback on their progress or health 61D. In an embodiment, the subjection feedback may be a pain index where the seminar or training modules are directed to reducing a user's physical pain. The webpage 52D may employ a various pain score indexes for different health issues including the Visual Analog Scale (VAS), the Verbal Numerical Rating Scale (VNRS); the Verbal Descriptor Scale (VDS); the Brief Pain Inventory, and the Oswestry Disability Index (ODI). A DTS 40A may employ a user's subjective feedback to map their progress, update their prognosis, and potentially change their calendar or suggested activities.

In an embodiment, a DTS 40A may combine a user's subjective data with user UAMM data to determine the user's progress and prognosis. A user's subjective data or activity data may be weighted based on similar users and the related completed or attempted seminars. The subjective and activity data may also be variably weighted based on their differential levels between the user data and related users. A user's UAMM may also be used to update their suggested activities (calendar) and progress (activity 184B of process 170B, FIG. 6B).

FIG. 4C is a block diagram of dynamic training system architecture 130C providing a media display web page 52C according to various embodiments. As shown in FIG. 4C, a DTS 40A may provide media to a UHD 12A via a webpage 52C. The media may include one or more seminar or training modules 310A-J. The DTS 40A may format the media based on the UHD 12A-E media display capabilities, user selection, or web request from the UHD 12A-E. The media 53C may include video, audio, text, pictures, or a combination thereof. A user 136 via the control menu 51C may be able to control the media presentation and format. In an embodiment, a DTS 40A may monitor a user UAMM 13A-13C while a user is receiving media content to ensure compliance and develop metrics for related users. The metrics may include movement levels, movement types, and biometric data during the media presentation. A DTS 40A may modify or update a user's prognosis, progress, and calendar based on their measured activity during a seminar or training module. A DTS 40A may compare a user's metrics to users having similar demographics for similar or same seminars or training modules 310A-J.

FIG. 7A illustrates a block diagram of a device 230 that may be employed at least in part in a UHD 12A-12E or UAMM 13A-C in various embodiments. The device 230 may include a central processing unit (CPU) 232, a random access memory (RAM) 234, a read only memory (ROM) 237, a local wireless/GPS modem/transceiver 244, an accelerometer 235, a display 247, a camera 256, a speaker 245, a rechargeable electrical storage element 256, and an antenna 246. The CPU 232 may include a control interface 254 including an IP type network controller interface (MC). The RAM 234 may include a queue or table 248 where the queue 248 may be used to store web pages. The RAM 234 may also include program, algorithm, and system data and instructions. The rechargeable electrical storage element may be a battery or capacitor in an embodiment.

The modem/transceiver 244 may couple, in a well-known manner, the device 230 to a wired or wireless network 30A, 30B to enable communication with a DTS 40A-40B, UHD 12A-E, or UAMM 13A-13C. The modem/transceiver 244 may also be able to receive global positioning signals (GPS) and the CPU 232 may be able to convert the GPS signals to location data that may be stored in the RAM 234 and provide the GPS data and accelerometer data to a DTS 40A-40B. The ROM 237 may store program instructions to be executed by the CPU 232 or control interface 254 (applications 237A). The applications 237A may include a web browser program or application. The RAM 234 may also be used to store program information, queues, databases, and overhead information.

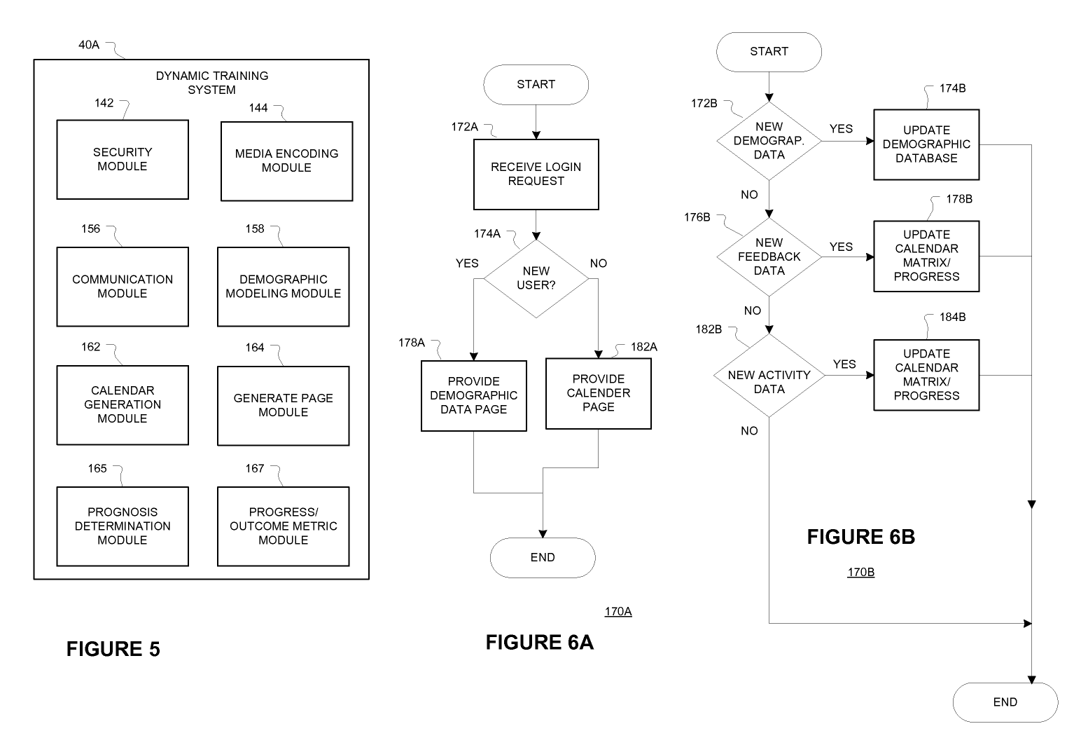

FIG. 5 is a block diagram of a dynamic training system 40A modules according to various embodiments. As shown in FIG. 5, a DTS 40A may include a security module 142, a media encoding module 144, a communication module 156, a demographic modeling module 158, a calendar generation module 162, a generate web page module 164, a prognosis determination module 165, and a progress/outcome metric module 167. The security module 142 may verify a user's access to a DTS 40A, 40B and particular seminars and training modules 310A-J. The media encoding module may transcode media to be communicated to a UHD 12A-12E. The communication module 156 may enable wired, wireless, and direct communication between a DTS 40A and another DTS 40B, central DTS 60A, UHD 12A-12E, and UAMM 13A-13C. The demographic modeling module 158 may store and categorize user demographic data with related users.

The categorized data may be employed by other DTS modules including the prognosis determination module 165, the calendar generation module 162, and the progress/outcome metric module 167. The calendar generation module 162 may generate a user seminar/training module calendar based on user demographics, seminar selections, activity monitoring, and user feedback. The generate page module 164 may generate web pages for use by a UHD 12A-12E web browser or other control device 60A. The prognosis determination module 165 may use the user demographics, related user demographics, user activity data and feedback, and elected and completed seminars/training modules 310A-J. The progress/outcome metric module may determine a user's progress or outcome based on completed seminars, user activity data, and user feedback data.

FIG. 7B illustrates a block diagram of a device 260 that may be employed at least in part in a DTS 40A-40B, 60A in various embodiments. The device 260 may include a central processing unit (CPU) 262, a random access memory (RAM) 264, a read only memory (ROM) 266, a display 268, a user input device 272, a transceiver application specific integrated circuit (ASIC) 274, a microphone 288, a speaker 282, storage 276, electrical energy storage unit 286, and an antenna 284. The CPU 262 may include a server 292. The RAM 264 may include a queue 278 where the queue 278 may store media. The server 292 may function as the web-server/e-mail processor 42A, 42B of the DTS 40A, 40B.

The ROM 266 is coupled to the CPU 262 and may store the program instructions to be executed by the CPU 262 and the server 292. The ROM 266 may include applications and instructions for the webserver 42A, media parser 44, web server 46, security module 142, media encoding module 144, IP communication module 156, reference image generation module 158, local wireless communication module 162, and generate page module 164. The RAM 264 may be coupled to the CPU 262 and may store temporary program data, overhead information, and the queues 278. The user input device 272 may comprise an input device such as a keypad, touch pad screen, track ball or other similar input device that allows the user to navigate through menus in order to operate the device 260. The display 268 may be an output device such as a CRT, LCD or other similar screen display that enables the user to read, view, or hear multimedia content.

The microphone 288 and speaker 282 may be incorporated into the device 260. The microphone 288 and speaker 282 may also be separated from the device 260. Received data may be transmitted to the CPU 262 via a serial bus 275 where the data may include messages, user data, or pages received, messages, digital media content associated with seminars, or web pages to be transmitted, or protocol information. The transceiver ASIC 274 may include an instruction set necessary to communicate messages or web pages via network 30A, 30B. The ASIC 274 may be coupled to the antenna 284 to communicate messages, content, or pages wireless. When a message is received by the transceiver ASIC 274, its corresponding data may be transferred to the CPU 262 via the serial bus 276. The data can include wireless protocol, overhead information, sensor, and pages to be processed by the device 260 in accordance with the methods described herein.

The rechargeable electrical storage element 286 may be a battery or capacitor in an embodiment. The storage 276 may be any digital storage medium and may be coupled to the CPU 262 and may store temporary program data, overhead information, and databases 48, 49.

Any of the components previously described can be implemented in a number of ways, including embodiments in software. Any of the components previously described can be implemented in a number of ways, including embodiments in software. Thus, the devices 230, 260 elements including the RAM 234, ROM 237, CPU 232, transceiver 244, storage 276, CPU 262, RAM 264, ROM 266, and transceiver ASIC 274, may all be characterized as “modules” herein.

The modules may include hardware circuitry, single or multi-processor circuits, memory circuits, software program modules and objects, firmware, and combinations thereof, as desired by the architect of the architecture 10 and as appropriate for particular implementations of various embodiments.

The apparatus and systems of various embodiments may be useful in applications other than a sales architecture configuration. They are not intended to serve as a complete description of all the elements and features of apparatus and systems that might make use of the structures described herein.

Applications that may include the novel apparatus and systems of various embodiments include electronic circuitry used in high-speed computers, communication and signal processing circuitry, modems, single or multi-processor modules, single or multiple embedded processors, data switches, and application-specific modules, including multilayer, multi-chip modules. Such apparatus and systems may further be included as sub-components within a variety of electronic systems, such as televisions, cellular telephones, personal computers (e.g., laptop computers, desktop computers, handheld computers, tablet computers, etc.), workstations, radios, video players, audio players (e.g., mp3 players), vehicles, medical devices (e.g., heart monitor, blood pressure monitor, etc.) and others. Some embodiments may include a number of methods.

It may be possible to execute the activities described herein in an order other than the order described. Various activities described with respect to the methods identified herein can be executed in repetitive, serial, or parallel fashion.

A software program may be launched from a computer-readable medium in a computer-based system to execute functions defined in the software program. Various programming languages may be employed to create software programs designed to implement and perform the methods disclosed herein. The programs may be structured in an object-orientated format using an object-oriented language such as Java or C++. Alternatively, the programs may be structured in a procedure-orientated format using a procedural language, such as assembly or C. The software components may communicate using a number of mechanisms well known to those skilled in the art, such as application program interfaces or inter-process communication techniques, including remote procedure calls. The teachings of various embodiments are not limited to any particular programming language or environment.

The accompanying drawings that form a part hereof show, by way of illustration and not of limitation, specific embodiments in which the subject matter may be practiced. The embodiments illustrated are described in sufficient detail to enable those skilled in the art to practice the teachings disclosed herein. Other embodiments may be utilized and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

The Abstract of the Disclosure is provided to comply with 37 C.F.R. § 1.72(b), requiring an abstract that will allow the reader to quickly ascertain the nature of the technical disclosure. It is submitted with the understanding that it will not be used to interpret or limit the scope or meaning of the claims. In the foregoing Detailed Description, various features are grouped together in a single embodiment for the purpose of streamlining the disclosure. This method of disclosure is not to be interpreted to require more features than are expressly recited in each claim. Rather, inventive subject matter may be found in less than all features of a single disclosed embodiment. Thus the following claims are hereby incorporated into the Detailed Description, with each claim standing on its own as a separate embodiment.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. A system for dynamically providing physical training to a user, including:
a content delivery module, the module including:
a digital media database including a plurality of seminars, each seminar including at least one exercise to be completed by a user; and
a digital media streaming module, the streaming module streaming in real time one of the plurality of seminars;
a user demographic database including a plurality of user demographic data values for each user of the system;
a feedback module, the feedback module electronically collecting a plurality of user feedback data and storing the collected data in a database;
a training calendar database including a plurality of personalized training calendars, each training calendar including a plurality of events, each event including a seminar to be performed and a schedule for the seminar performance; and
a calendar generation module, the module generating a personalized training calendar from the training calendar database for a user based on the user demographic database, the feedback data in a database, and at least one demographic data value for the user.

24. The system for providing training to a user of claim 23, wherein the feedback data in a database includes user data collected from a plurality of users during the streaming of each seminar and the feedback module storing a plurality of user data during the streaming of a seminar.

25. The system for providing training to a user of claim 23, wherein the feedback data in a database includes user data collected electronically from a plurality of users during the streaming of each seminar and after the streaming of each seminar and the feedback module electronically collecting a plurality of user data during the streaming of a seminar and after the streaming of a seminar.

26. The system for providing training to a user of claim 23, wherein each training calendar includes a plurality of ordered events.

27. The system for providing training to a user of claim 24, wherein the feedback data includes user objective and subjective data.

28. The system for providing training to a user of claim 27, wherein the objective data is sensor based objective activity data.

29. The system for providing training to a use of claim 23, wherein the seminar includes a plurality of exercises directed to improving a specific user physical condition.

30. The system for providing training to a user of claim 23, wherein the seminar includes a plurality of exercises directed to one of reducing or improving pain and the feedback data includes pain levels.

31. The system for providing training to a user of claim 28, wherein the sensor based objective activity data of a user include one of heart rate, blood pressure, and glucose levels.

32. The system for providing training to a user of claim 27, wherein the subjective data includes pain frequency measurements.

33. The system for providing training to a user of claim 27, wherein the subjective data includes pain frequency and intensity.

34. The system for providing training to a user of claim 28, wherein the seminar includes a plurality of exercises directed to one of reducing or improving pain and the feedback data includes activity levels determined by the biometric sensors.

35. The system for providing training to a use of claim 28, wherein the calendar generation module generates a personalized training calendar from the training calendar database based in part on the sensor based objective activity data.

36. The system for providing training to a use of claim 28, wherein the calendar generation module generates a personalized training calendar from the training calendar database based in part on the sensor based objective activity data for a user as compared to other users' sensor based objective activity data.

37. The system for providing training to a user of claim 23, wherein user demographic data values includes one of age, weight, height, geographical location, race, and health issues.

38. The system for providing training to a user of claim 23, further comprising a prognosis determination module, the prognosis determination module updating a user outcome database based on feedback data in a database and the demographic database for a plurality of users, the user outcome database including outcome data for a plurality of users in the user demographic database.

39. The system for providing training to a user of claim 38, wherein the prognosis determination module further generates a user's potential outcome based on the user outcome database, the demographic database, and the user's personalized training calendar.

40. The system for providing training to a user of claim 38, wherein the prognosis determination module further generates a user's potential outcome based on the user outcome database, the demographic database, the user's measured sensor based objective activity data, and the user's personalized training calendar.

41. The system for providing training to a user of claim 23, wherein the feedback module determines a user's compliance based on the feedback data in a database.

42. The system for providing training to a user of claim 23, further comprising a prognosis determination module, the prognosis determination module generating a user outcome prediction based on a user outcome database, the demographic database, and at least one of the user's demographic data, the user outcome database including outcome data for a plurality of users in the user demographic database.
